SEQUENCE LISTING (3 sequences)

SEQ ID NO: 1 - DNA, 17 bp, Homo sapiens
cgcagactcgagcccgg

SEQ ID NO: 2 - DNA, 32 bp, Homo sapiens
ccgccccctctggcagcacggaaacctccacg

SEQ ID NO: 3 - DNA, 24 bp, Homo Sapiens
ggcgtcacaaaccccggagagcgg

(19) United States
(12) Patent Application Publication
(10) Pub. No.: US 2016/0228496 A1
(43) Pub. Date: Aug. 11, 2016

(54) METHODS FOR TREATING HCV

(71) Applicant: AbbVie Inc., North Chicago, IL (US)

(72) Inventors: Daniel E. Cohen, North Chicago, IL (US); Kenneth B. Idler, Trevor, WI (US); Jeffrey F. Waring, Franklin, WI (US); Emily O. Dumas, Libertyville, IL (US); Sandeep Dutta, Lincolnshire, IL (US)

(73) Assignee: AbbVie Inc., North Chicago, IL (US)

(21) Appl. No.: 14/208,266

(22) Filed: Mar. 13, 2014

Related U.S. Application Data
(60) Provisional application No. 61/858,960, filed on Jul. 26, 2013.
(60) Provisional application No. 61/791,840, filed on Mar. 15, 2013.

(51) Int. Cl. A61K 38/12; A61K 38/05; C12Q 1/68; A61K 31/513

(52) U.S. Cl. CPC A61K 38/12; A61K 31/513; A61K 38/05; C12Q 1/6883

(57) ABSTRACT

The present invention features therapies for the treatment of HCV comprising direct-acting antiviral agents. Preferably, the treatment is administered to an HCV-infected patient who has been tested to determine methylation status of a CpG island within a promoter region of the IL28B gene. In one aspect, the therapies comprise administering one or more direct acting antiviral agents and, optionally ribavirin, to a subject with HCV infection. For example, the therapies comprise administering to the subject effective amounts of therapeutic agent 1, therapeutic agent 2, therapeutic agent 3, an inhibitor of cytochrome P450 (e.g., ritonavir), and/or ribavirin.

### CROSS-REFERENCE TO RELATED APPLICATIONS

This application claims priority to U.S. provisional application No. 61/791,840, filed on Mar. 15, 2013 and U.S. provisional application No. 61/858,960, filed on Jul. 26, 2013, each of which are incorporated by reference in their entireties.

### FIELD OF THE INVENTION

The present invention relates to treatment for hepatitis C virus (HCV) using a direct-acting antiviral regimen. The present invention relates to methylation status of CpG islands in the promoter region of the IL28B gene as diagnostic and prognostic markers for HCV.

### BACKGROUND OF THE INVENTION

HCV is an RNA virus belonging to the Hepacivirus. HCV contains approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

Chronic HCV infection is associated with progressive liver pathology, including cirrhosis and hepatocellular carcinoma. Chronic HCV infection is characterized by high inter-individual variability in terms of response to currently approved treatments. Chronic HCV infection may be treated by peginterferon-alpha (PEG-IFNα) in combination with ribavirin and with or without one of the recently approved linear protease inhibitors, VX-950 (telaprevir) or SCHSO3034 (boceprevir). Substantial limitations to efficacy and tolerability remain as many users suffer from side effects, and viral elimination from the body is often incomplete even after such treatments. Therefore, there is a need for new therapies to treat HCV infection and a need to identify patients who will benefit from particular therapeutic regimens.

CpG islands are stretches of DNA in which the frequency of a cytosine next to a guanine is higher than in other regions of the genome. CpG islands are generally located within 5′ promoter regions of genes. Cytosines in CpG dinucleotides can be methylated to form 5-methylcytosine. It is believed that methylation of CpG sites in the promoter region of a gene may inhibit gene expression. The degree of methylation may determine the extent of active transcription.

Interleukin 28B (“IL28B”), or interferon lambda 3, is a gene mapped to chromosome 19. IL28B has three CpG islands in the 5′ promoter region, and each island has three potential cytosine methylation sites. The sites are proximal to single polynucleotide polymorphism (SNP) of IL28B, rs12979860, that is somewhat predictive of the response of HCV-infected subjects to interferon treatment.

### BRIEF SUMMARY OF THE INVENTION

It was unexpectedly discovered that patients who failed standard of care treatment have higher methylation levels in a CpG island in a promoter region of the IL28B gene. Thus, methylation status of a CpG island in a promoter region of the IL28B gene can be used as a biomarker for individuals that may relapse or otherwise inadequately respond to treatment with standard of care therapy. Such information can be used to guide therapy.

It was unexpectedly discovered that HCV-infected patients had higher levels of methylation of at least one CpG island within an IL28B promoter region as compared to healthy subjects. Thus, methylation levels of a CpG island in an IL28B promoter region can be used as a biomarker for individuals that are infected with HCV. Such information can be used to confirm or establish diagnosis.

It was unexpectedly discovered that patients with an inadequate response following treatment with a direct-acting antiviral regimen for 8 weeks had higher levels of methylation of a CpG island within an IL28B promoter region as compared to patients that showed an adequate response following treatment with a direct-acting antiviral regimen for 8 weeks. Thus, methylation levels of a CpG island in an IL28B promoter region can be used to select an appropriate treatment duration for individuals that are infected with HCV. Such information can be used to guide therapy. In particular, such information can be used to select a duration of therapy.

At least one aspect of the present invention provides methods for treating a subject infected with Hepatitis C virus with a direct-acting antiviral regimen. The methods comprise administering the direct-acting antiviral regimen to the subject; wherein prior to the administration of the direct-acting antiviral regimen a DNA sample from the patient has been tested to determine methylation status of at least one CpG island in an IL28B promoter region.

At least one aspect of the present invention provides kits to predict responsiveness of a subject to treatment of hepatitis C infection. The kits comprise an assay for detecting methylation of at least one CpG island in a promoter region of an IL28B gene; and a written instruction that states a level of hypermethylation of the CpG island that predicts an inadequate response to treatment.

At least one aspect of the present invention provides methods for detecting a risk of treatment failure in a Hepatitis C infected subject. The methods comprise (a) providing a sample comprising a promoter region of the IL28B gene from the Hepatitis C infected subject; and (b) detecting methylation status of at least one CpG island in the promoter region of the IL28B gene from the Hepatitis C infected subject. In accordance with these methods, hypermethylation of the CpG island in the promoter region is indicative of a risk for treatment failure.

At least one aspect of the present invention provides methods for classifying a subject infected with hepatitis C as a candidate to receive direct-acting antiviral therapy. The methods comprise: (a) providing a biological sample from the subject, the sample comprising a promoter region of the IL28B gene; (b) detecting methylation status of at least one CpG island in the promoter region of the IL28B gene; and (c) classifying the patient as eligible to receive direct-acting antiviral therapy based on the methylation status of the CpG island.

At least one aspect of the present invention provides methods for detecting sensitivity of a subject to treatment for Hepatitis C infection. The methods comprise: (a) providing a DNA sample from the subject, the sample comprising a non-coding region of the IL28B gene; (b) determining methylation status of at least one CpG island in the non-coding region of an IL28B gene, wherein hypermethylation of the CpG island in the non-coding region is indicative of the subject being at least partially insensitive to treatment with an interferon-containing regimen; and (c) administering a direct-acting antiviral regimen to the subject.

At least one aspect of the present invention provides methods to treat a patient infected with Hepatitis C virus with a direct-acting antiviral regimen. The methods comprise administering the direct-acting antiviral regimen to the patient. In the present methods, prior to the administration of the direct-acting antiviral regimen a sample obtained from the patient has been tested to determine a methylation level of a promoter region of an IL28B gene. In some embodiments, the methylation level is determined for a CpG island located within the promoter region of the IL28B gene. In some embodiments, the CpG island is SEQ ID NO:1, SEQ ID NO:2, or SEQ ID NO:3. In some embodiments, the methylation level is greater than a mean methylation level in a population of HCV patients. In some embodiments, the methylation level is greater than a mean methylation level in a population of HCV patients who achieve a sustained virological response following treatment with an interferon-containing regimen. In some embodiments, the methylation level is greater than an established cut-off value. The cut-off value may be, for example, 35%, 40%, 45%, 50%, or 55%. In certain embodiments, the methylation level is greater than 40%. In certain embodiments, the methylation level is greater than 45%. In some embodiments, the sample is a blood sample, such as a sample containing white blood cells. In some embodiments, the direct-acting antiviral regimen comprises one or more direct acting antiviral agents (DAAs). In some embodiments, the methods further comprise administering an inhibitor of cytochrome P-450 (such as ritonavir) to the patient to improve the pharmacokinetics or bioavailability of one or more of the DAAs. In some embodiments, the components of the direct-acting antiviral regimen are administered in amounts effective to provide a sustained virological response (SVR) or achieve another desired measure of effectiveness in a patient.

At least one aspect of the present invention provides methods to treat a patient infected with Hepatitis C virus with a direct-acting antiviral regimen. The methods comprise administering the direct-acting antiviral regimen to the patient. In the present methods, prior to the administration of the direct-acting antiviral regimen a sample obtained from the patient has been tested to determine a methylation level of a promoter region of an IL28B gene. In some embodiments, the methylation level is determined for a CpG island located within the promoter region of the IL28B gene. In some embodiments, the CpG island is SEQ ID NO:1, SEQ ID NO:2, or SEQ ID NO:3. In some embodiments, the methylation level is greater than a pre-determined control level. The pre-determined control level can be a range or a specific value. The pre-determined control level can be a percentage of methylation, for example, 35%, 40%, 45%, 50%, or 55%. In certain embodiments, the methylation level is greater than 40%. In certain embodiments, the methylation level is greater than 45%. The pre-determined control level can be determined empirically, such as, by obtaining a mean methylation level from a population of subjects. The population of subjects can be a population of healthy subjects or a population of HCV-infected subjects. Thus, the pre-determined control level can be, for example, a mean methylation level from a population of patients infected with Hepatitis C virus. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. In some embodiments, the components of the direct-acting antiviral regimen are administered in amounts effective to provide an SVR or achieve another desired measure of effectiveness in a patient.

At least one aspect of the present invention provides methods to treat a patient infected with Hepatitis C virus with a direct-acting antiviral regimen for no more than 8 weeks. The methods comprise administering the direct-acting antiviral regimen to the patient for no more than 8 weeks. In some embodiments, the methods may comprise administering the direct-acting antiviral regimen to the patient for 8 weeks. In the present methods, prior to the administration of the direct-acting antiviral regimen a sample obtained from the patient has been tested to determine a methylation level of a promoter region of an IL28B gene. In some embodiments, the methylation level is determined for a CpG island located within the promoter region of the IL28B gene. In some embodiments, the CpG island is SEQ ID NO:1, SEQ ID NO:2, or SEQ ID NO:3. In some embodiments, the patient is a selected patient, where the selection was based on the determined methylation level of the promoter region of the IL28B gene. In some embodiments, the methylation level of the selected patient is less than or equal to a pre-determined control level. The pre-determined control level can be a range or a specific value. The pre-determined control level can be a percentage of methylation, for example, 35%, 40%, 45%, 50%, or 55%. In certain embodiments, the methylation level is less than or equal to 40%. In certain embodiments, the methylation level is less than or equal to 45%. The pre-determined control level can be determined empirically, such as, by obtaining a mean methylation level from a population of subjects. The population of subjects can be a population of healthy subjects or a population of HCV-infected subjects. Thus, the pre-determined control level can be, for example, a mean methylation level from a population of patients infected with Hepatitis C virus. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. In some embodiments, the components of the direct-acting antiviral regimen are administered in amounts effective to provide an SVR or achieve another desired measure of effectiveness in a patient.

At least one aspect of the present invention provides methods to treat a patient infected with Hepatitis C virus with a direct-acting antiviral regimen for more than 8 weeks. The methods comprise administering the direct-acting antiviral regimen to the patient for more than 8 weeks. In some embodiment, the duration of treatment with the direct-acting antiviral regimen is 12 weeks. In the present methods, prior to the administration of the direct-acting antiviral regimen a sample obtained from the patient has been tested to determine a methylation level of a promoter region of an IL28B gene. In some embodiments, the methylation level is determined for a CpG island located within the promoter region of the IL28B gene. In some embodiments, the CpG island is SEQ ID NO:1, SEQ ID NO:2, or SEQ ID NO:3. In some embodiments, the patient is a selected patient, where the selection was based on the determined methylation level of the promoter region of the IL28B gene. In some embodiments, the methylation level of the selected patient is greater than a pre-determined control level. The pre-determined control level can be a range or a specific value. The pre-determined control level can be a percentage of methylation, for example, 35%, 40%, 45%, 50%, or 55%. In certain embodiments, the methylation level is greater than 40%. In certain embodiments, the methylation level is greater than 45%. The pre-determined control level can be determined empirically, such as, by obtaining a mean methylation level from a population of subjects. The population of subjects can be a population of healthy subjects or a population of HCV-infected subjects. Thus, the pre-determined control level can be, for example, a mean methylation level from a population of patients infected with Hepatitis C virus. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. In some embodiments, the components of the direct-acting antiviral regimen are administered in amounts effective to provide an SVR or achieve another desired measure of effectiveness in a patient.

At least one aspect of the present invention provides methods to treat a patient infected with Hepatitis C virus. The methods comprise identifying a patient having a level of methylation within a promoter region of an IL28B gene that is predictive of failing to achieve a sustained response to an interferon-containing regimen; selecting the identified patient for treatment with a direct-acting antiviral regimen; and administering the direct-acting antiviral regimen to the selected patient. In some embodiments, the methylation level that is predictive of failing to achieve a sustained response to an interferon-containing regimen is greater than or equal to 40% methylation. In some embodiments, the methylation level that is predictive of failing to achieve a sustained response to an interferon-containing regimen is greater than or equal to 45% methylation. In some embodiments, the methylation level in the patient sample is greater than a pre-determined control level. In some embodiments, the pre-determined control level is a mean methylation level from a population of patients infected with Hepatitis C virus. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. In some embodiments, the components of the direct-acting antiviral regimen are administered in amounts effective to provide an SVR or achieve another desired measure of effectiveness in a subject.

At least one aspect of the present invention provides methods to treat a patient infected with Hepatitis C virus. The methods comprise identifying a patient having a level of methylation within a promoter region of an IL28B gene that is predictive of failing to achieve a sustained response to a direct-acting antiviral regimen administered for 8 weeks; selecting the identified patient for treatment with a direct-acting antiviral regimen for more than 8 weeks; and administering the direct-acting antiviral regimen to the selected patient for more than 8 weeks. In some embodiments, the duration of treatment with the direct-acting antiviral regimen is 12 weeks. In some embodiments, the methylation level that is predictive of failing to achieve a sustained response to a direct-acting antiviral regimen administered for 8 weeks is greater than 40% methylation. In some embodiments, the methylation level that is predictive of failing to achieve a sustained response to a direct-acting antiviral regimen administered for 8 weeks is greater than 45% methylation. In some embodiments, the methylation level in the patient sample is greater than a pre-determined control level. In some embodiments, the pre-determined control level is a mean methylation level from a population of patients infected with Hepatitis C virus. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. In some embodiments, the components of the direct-acting antiviral regimen are administered in amounts effective to provide an SVR or achieve another desired measure of effectiveness in a subject.

At least one aspect of the present invention provides methods to treat a patient infected with Hepatitis C virus. The methods comprise identifying a patient having a level of methylation within a promoter region of an IL28B gene that is predictive of achieving a sustained response to a direct-acting antiviral regimen administered for no more than 8 weeks; selecting the identified patient for treatment with a direct-acting antiviral regimen for no more than 8 weeks; and administering the direct-acting antiviral regimen to the selected patient for no more than 8 weeks. In some embodiments, the direct-acting antiviral regimen is administered to the selected patient for 8 weeks. In some embodiments, the methylation level that is predictive of achieving a sustained response to a direct-acting antiviral regimen administered for no more than 8 weeks is less than or equal to 40% methylation. In some embodiments, the methylation level that is predictive of achieving a sustained response to a direct-acting antiviral regimen administered for no more than 8 weeks is less than or equal to 45% methylation. In some embodiments, the methylation level in the patient sample is less than or equal to a pre-determined control level. In some embodiments, the pre-determined control level is a mean methylation level from a population of patients infected with Hepatitis C virus. In some embodiments, the direct-acting antiviral regimen comprises one or more DAAs. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. In some embodiments, the components of the direct-acting antiviral regimen are administered in amounts effective to provide an SVR or achieve another desired measure of effectiveness in a subject.

At least one aspect of the present invention provides methods to treat a patient having a Hepatitis C virus infection with a direct-acting antiviral regimen. The methods comprise assessing methylation status of a CpG island within a promoter region of an IL28B gene in a sample obtained from a patient infected with Hepatitis C virus to obtain a patient-specific methylation level; predicting responsiveness to an interferon-containing regimen, wherein a patient-specific methylation level that is greater than a pre-determined control level is predictive of the patient failing to achieve a sustained response to the interferon-containing regimen; and administering the direct-acting antiviral regimen to the patient based upon the methylation level. In some embodiments, the pre-determined control level is a percentage of methylation. In some embodiments, the pre-determined control level is greater than or equal to 40% methylation. In some embodiments, the pre-determined control level is greater than or equal to 45% methylation. In some embodiments, the pre-determined control level is a mean methylation level from a population of patients infected with Hepatitis C virus. In some embodiments, the sample is a blood sample, such as a sample containing white blood cells. In some embodiments, the direct-acting antiviral regimen comprises at least two DAAs, with or without ribavirin. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. In some embodiments, the components of the direct-acting antiviral regimen are administered in amounts effective to provide an SVR or achieve another desired measure of effectiveness in a patient.

At least one aspect of the present invention provides methods to treat a patient having a Hepatitis C virus infection with a direct-acting antiviral regimen. The methods comprise assessing methylation status of a CpG island within a promoter region of an IL28B gene in a sample obtained from a patient infected with Hepatitis C virus to obtain a patient-specific methylation level; predicting responsiveness to a direct-acting antiviral regimen administered for 8 weeks, wherein a patient-specific methylation level that is greater than a pre-determined control level is predictive of the patient failing to achieve a sustained response to direct-acting antiviral regimen administered for 8 weeks; and administering the direct-acting antiviral regimen to the patient for more than 8 weeks based upon the methylation level. In some embodiments, the pre-determined control level is a percentage of methylation. In some embodiments, the pre-determined control level is greater than 40% methylation. In some embodiments, the pre-determined control level is greater than 45% methylation. In some embodiments, the pre-determined control level is a mean methylation level from a population of patients infected with Hepatitis C virus. In some embodiments, the sample is a blood sample, such as a sample containing white blood cells. In some embodiments, the direct-acting antiviral regimen comprises at least two DAAs, with or without ribavirin. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. In some embodiments, the components of the direct-acting antiviral regimen are administered in amounts effective to provide an SVR or achieve another desired measure of effectiveness in a patient.

At least one aspect of the present invention provides methods to treat a patient having a Hepatitis C virus infection with a direct-acting antiviral regimen. The methods comprise assessing methylation status of a CpG island within a promoter region of an IL28B gene in a sample obtained from a patient infected with Hepatitis C virus to obtain a patient-specific methylation level; predicting responsiveness to a direct-acting antiviral regimen administered for no more than 8 weeks, wherein a patient-specific methylation level that is less than or equal to a pre-determined control level is predictive of the patient achieving a sustained response to direct-acting antiviral regimen administered for no more than 8 weeks; and administering the direct-acting antiviral regimen to the patient for no more than 8 weeks based upon the methylation level. In some embodiments, the direct-acting antiviral regimen is administered to the patient for 8 weeks. In some embodiments, the pre-determined control level is a percentage of methylation. In some embodiments, the pre-determined control level is less than or equal to 40% methylation. In some embodiments, the pre-determined control level is less than or equal to 45% methylation. In some embodiments, the pre-determined control level is a mean methylation level from a population of patients infected with Hepatitis C virus. In some embodiments, the sample is a blood sample, such as a sample containing white blood cells. In some embodiments, the direct-acting antiviral regimen comprises at least two DAAs, with or without ribavirin. In some embodiments, the methods further comprise administering an inhibitor of cytochrome P-450 (such as ritonavir) to the patient to improve the pharmacokinetics or bioavailability of one or more of the DAAs. In some embodiments, the direct-acting antiviral regimen comprises an HCV protease inhibitor and an HCV polymerase inhibitor. In some embodiments, the components of the direct-acting antiviral regimen are administered in amounts effective to provide an SVR or achieve another desired measure of effectiveness in a patient.

At least one aspect of the present invention provides methods for determining a treatment regimen for a subject infected with hepatitis C as a candidate to receive a direct-acting antiviral regimen for no more than 8 weeks. The methods comprise providing a biological sample from the subject, the sample comprising a promoter region of an IL28B gene; detecting methylation status of at least one CpG island in the promoter region of the IL28B gene; and classifying the patient as eligible to receive the direct-acting antiviral regimen for no more than 8 weeks based on the methylation status of the CpG island. In certain embodiments, the CpG island comprises SEQ ID NO:1, SEQ ID NO:2, or SEQ ID NO:3. In certain embodiments, the methods further comprise administering a direct-acting antiviral regimen to the subject for no more than 8 weeks. In certain embodiments, the direct-acting antiviral regimen comprises an HCV protease inhibitor, an HCV polymerase inhibitor, and an HCV NS5A inhibitor. In certain embodiments, the direct-acting antiviral regimen comprises therapeutic agent 1, therapeutic agent 2, and therapeutic agent 3.

At least one aspect of the present invention provides methods for treating a subject with Hepatitis C infection. The methods comprise providing a DNA sample from the subject, the sample comprising a non-coding region of an IL28B gene; (b) determining methylation status of at least one CpG island in the non-coding region of the IL28B gene, wherein hypermethylation of the CpG island in the non-coding region is indicative of the subject being at least partially insensitive to an 8 week course of treatment with a direct acting antiviral regimen; and (c) administering a direct-acting antiviral regimen to the subject for more than 8 weeks. In certain embodiments, the CpG island comprises SEQ ID NO:1, SEQ ID NO:2, or SEQ ID NO:3. In certain embodiments, the direct-acting antiviral regimen comprises an HCV protease inhibitor, an HCV polymerase inhibitor, and an HCV NS5A inhibitor. In certain embodiments, the direct-acting antiviral regimen comprises therapeutic agent 1, therapeutic agent 2, and therapeutic agent 3.

At least one aspect of the present invention provides methods for treating HCV infection in a patient who has been tested to establish a level of methylation within a promoter region of an IL28B gene. The methods comprise administering a protease inhibitor and one or more polymerase inhibitors to the patient. The protease inhibitor can be therapeutic agent 1. The polymerase inhibitor can be therapeutic agent 2. The methods also can comprise administering ribavirin and/or an inhibitor of cytochrome P-450 to the patient. The methods also can comprise administering an NS5A inhibitor to the patient. The NS5A inhibitor can be therapeutic agent 3. In some embodiments, the protease inhibitor, the polymerase inhibitor(s), the NS5A inhibitor, ribavirin and/or the inhibitor of cytochrome P-450 are administered in amounts effective to provide SVR or another measure of effectiveness in the patient. As non-limiting examples, therapeutic agent 1, therapeutic agent 3, and the inhibitor of cytochrome P-450 can be co-formulated and administered once daily, and therapeutic agent 2 can be administered twice daily.

In the foregoing methods as well as methods described herein, the methods can include administering ribavirin. The methods also can include administering ritonavir or another CYP3A4 inhibitor (e.g., cobicistat) if one of the DAAs requires or benefits from pharmacokinetic enhancement. Where the direct-acting antiviral regimen comprises at least two DAAs, the at least two DAAs can be administered concurrently or sequentially. For example, one DAA can be administered once daily, and another DAA can be administered twice daily. As a non-limiting example, the patient being treated can be infected with HCV genotype 1, such as genotype 1a or 1b. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder, a partial responder or a relapser), or not a candidate for interferon treatment.

In the foregoing methods as well as methods described hereinbelow, the direct-acting antiviral regimen can comprise protease inhibitors, nucleoside or nucleotide polymerase inhibitors, non-nucleoside polymerase inhibitors, NS3B inhibitors, NS4A inhibitors, NS5A inhibitors, NS5B inhibitors, cyclophilin inhibitors, and combinations of any of the foregoing. For example, in some embodiments, the direct-acting antiviral regimen can comprise or consist of at least one HCV protease inhibitor and at least one HCV polymerase inhibitor. The HCV polymerase inhibitor can be a nucleotide or nucleoside polymerase inhibitor. The HCV polymerase inhibitor can also be a non-nucleotide or non-nucleoside polymerase inhibitor.

In the foregoing methods as well as methods described hereinbelow, the DAAs can be selected from the group consisting of protease inhibitors, nucleoside or nucleotide polymerase inhibitors, non-nucleoside polymerase inhibitors, NS3B inhibitors, NS4A inhibitors, NS5A inhibitors, NS5B inhibitors, cyclophilin inhibitors, and combinations of any of the foregoing.

For example, in some embodiments, the DAAs used in the present methods comprise or consist of at least one HCV protease inhibitor and at least one HCV polymerase inhibitor. The HCV polymerase inhibitor can be a nucleotide or nucleoside polymerase inhibitor or a non-nucleoside polymerase inhibitor. The HCV polymerase inhibitor can also be a non-nucleotide polymerase inhibitor. In some embodiments, the HCV protease inhibitor is therapeutic agent 1 (described below) and the HCV polymerase inhibitor is therapeutic agent 2 (also described below). By way of example, therapeutic agent 1 can be administered at a total daily dose of 100 mg, alternatively 150 mg, alternatively 200 mg, or alternatively 250 mg. By way of example, therapeutic agent 1 can be administered in a total daily dose of from 100 mg to 250 mg, or administered at least once daily at a dose of from 150 mg to 250 mg, and therapeutic agent 2 can be administered twice daily at doses from 200 mg to 400 mg. For some embodiments, the HCV protease inhibitor is therapeutic agent 1 and the HCV polymerase inhibitor is a non-nucleos/tide polymerase inhibitor. Ritonavir (or another cytochrome P-450 3A4 inhibitor) can be co-administered or co-formulated with therapeutic agent 1 to improve the pharmacokinetics and bioavailability of therapeutic agent 1.

In some embodiments, the DAAs used in the present methods comprise or consist of at least one HCV protease inhibitor, at least one HCV polymerase inhibitor, and at least one NS5A inhibitor. The HCV polymerase inhibitor can be a nucleotide or nucleoside polymerase inhibitor. The HCV polymerase inhibitor can also be a non-nucleotide or non-nucleoside polymerase inhibitor. In some embodiments, the HCV protease inhibitor is therapeutic agent 1 (described below), the HCV polymerase inhibitor is therapeutic agent 2 (also described below), and the NS5A inhibitor is therapeutic agent 3 (also described below). By way of example, therapeutic agent 3 may be administered in a total daily dose amount of from 5 mg to 300 mg, or from 25 mg to 200 mg, or from 25 mg to 50 mg or any amounts there between. In some embodiments, the total daily dosage amount for therapeutic agent 3 is 25 mg. In some embodiments, therapeutic agent 3 is co-administered or co-formulated with therapeutic agent 1 and/or ritonavir (or another cytochrome P-450 3A4 inhibitor).

In the foregoing methods as well as methods described herein, a DAA can be administered in any effective dosing schemes and/or frequencies, for example, each DAA can be administered daily. Each DAA can be administered either separately or in combination, and each DAA can be administered at least once a day, at least twice a day, or at least three times a day. Likewise, the ribavirin can be administered at least once a day, at least twice a day, or at least three times a day, either separately or in combination with one or more of the DAAs. In some preferred embodiments, therapeutic agent 1 is administered once daily. In some preferred embodiments, therapeutic agent 2 is administered twice daily.

In some aspects, the direct-acting antiviral regimen can comprise (i) Compound 1 or a pharmaceutically acceptable salt thereof, which is co-administered or co-formulated with ritonavir; and (ii) Compound 2 or a pharmaceutically acceptable salt thereof. In some embodiments, the regimen can also comprise Compound 3 or a pharmaceutically acceptable salt thereof. In some embodiments, Compound 1 or a pharmaceutically acceptable salt thereof can be co-formulated with Compound 3 or a pharmaceutically acceptable salt thereof.

In another aspect, the present technology provides a combination of Compound 1 (or a pharmaceutically acceptable salt thereof), Compound 2 (or a pharmaceutically acceptable salt thereof), and/or Compound 3 (or a pharmaceutically acceptable salt thereof) for use in treating HCV infection. The treatment comprises administering the DAAs to an HCV-infected patient who has undergone testing to determine the methylation status of the promoter region of the IL28B gene prior to the treatment. The treatment includes administering ribavirin but does not include administering interferon. In some embodiments, ritonavir or another CYP3A4 inhibitor (e.g., cobicistat) is administered with Compound 1 (or the salt thereof) to improve the pharmacokinetics of the latter. Compound 1 (or the salt thereof), Compound 2 (or the salt thereof), and/or Compound 3 (or the salt thereof) can be administered concurrently or sequentially. For example, Compound 1 (or the salt thereof) can be administered once daily, together with ritonavir or another CYP3A4 inhibitor (e.g., cobicistat); Compound 2 (or the salt thereof) can be administered twice daily; and Compound 3 (or the salt thereof) can be administered once daily. For yet another example, Compound 1 (or the salt thereof) and ritonavir (or another CYP3A4 inhibitor, e.g., cobicistat) are co-formulated in a single composition and administered concurrently (e.g., once daily). For yet another example, Compound 1 (or the salt thereof), ritonavir (or another CYP3A4 inhibitor, e.g., cobicistat), and Compound 3 (or the salt thereof) are co-formulated in a single composition and administered concurrently (e.g., once daily). For yet another example, Compound 1 (or the salt thereof), co-formulated with ritonavir (or another CYP3A4 inhibitor, e.g., cobicistat) and/or Compound 3 (or the salt thereof), is administered once daily, and Compound 2 (or the salt thereof) is administered twice daily. As a non-limiting example, the patient being treated can be infected with HCV genotype 1, such as genotype 1a or 1b. As another non-limiting example, the patient can be infected with HCV genotype 2 or 3. As yet another non-limiting example, the patient can be a HCV-treatment naïve patient, a HCV-treatment experienced patient, an interferon non-responder (e.g., a null responder), or not a candidate for interferon treatment. The present technology also features the same aspect of the invention as described immediately above, except that the treatment does not include administering either ribavirin or interferon.

The direct-acting antiviral regimen of the present invention generally constitutes a complete treatment regimen, i.e., no subsequent interferon-containing regimen is intended. Thus, a treatment or use described herein generally does not include any subsequent interferon-containing treatment.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### BRIEF DESCRIPTION OF THE DRAWINGS

FIG. 1 is a plot showing percent methylation in three CpG islands in the promoter region of the IL28B gene. The three CpG islands are PM-01, PM-02, and PM-03. Patients are grouped as either treatment-naïve patients or treatment-experienced patients. The treatment-experienced patients previously received peg-interferon and ribavirin, but did not achieve an adequate sustained response. Data are plotted showing percent methylation for: CpG PM-01 in treatment-naïve patients (“Naïve 01”); CpG PM-01 in treatment-experienced patients (“Experienced 01”); CpG PM-02 in treatment-naïve patients (“Naïve 02”); CpG PM-02 in treatment-experienced patients (“Experienced 02”); CpG PM-03 in treatment-naïve patients (“Naïve 03”); and CpG PM-03 in treatment-experienced patients (“Experienced 03”).

FIG. 2 is a plot showing reduction in viral load in patients treated with peg-interferon and ribavirin over 10 days. Viral load was assessed on Day 1 (“D1”), Day 2 (“D2”), Day 3 (“D3”), Day 4 (“D4”), Day 5 (“D5”), and Day 10 (“D10”) of treatment. Patients are grouped by methylation status of CpG PM-02: greater than 45% methylation (diamonds; n=5) and less than 45% methylation (squares; n=6).

FIG. 3 is a plot showing reduction in viral load in patients treated with Compound 1 and ritonavir for 3 days followed by the addition of peginterferon and ribavirin for a total of 12 weeks of combination treatment. Viral load was assessed on Day 1 (“D1”), Day 2 (“D2”), Day 3 (“D3”), Day 4 (“D4”), Day 5 (“D5”), and Day 10 (“D10”) of treatment. Patients are grouped by methylation status of CpG PM-02: greater than 45% methylation (squares; n=10) and less than 45% methylation (diamonds; n=11).

FIGS. 4A, 4B, and 4C are a set of plots showing percent methylation in three CpG islands in the promoter region of the IL28B gene. FIG. 4A shows percent methylation of CpG PM-01. FIG. 4B shows percent methylation of CpG PM-02. FIG. 4C shows percent methylation of CpG PM-03. Patients are grouped by allelic status at a single IL28B SNP (rs12979860) as C/C (circles), C/T (triangles), or T/T (squares).

FIGS. 5A, 5B, and 5C are a set of plots showing percent methylation in CpG islands in the promoter region of the IL28B gene. Subjects were grouped as either healthy or HCV-infected. In FIG. 5A, three CpG islands are shown: PM-01, PM-02, and PM-03. Data are plotted showing percent methylation for: CpG PM-01 in healthy subjects (“PM-01 Healthy”); CpG PM-01 in HCV-infected subjects (“PM-01 HCV”); CpG PM-02 in healthy subjects (“PM-02 Healthy”); CpG PM-02 in HCV-infected subjects (“PM-02 HCV”); CpG PM-03 in healthy subjects (“PM-03 Healthy”); and CpG PM-03 in HCV-infected subjects (“PM-03 HCV”). In FIG. 5B, subjects are further grouped by genotype at rs12979860 as C/C, C/T, or T/T. Data are plotted showing percent methylation for PM-02 in: healthy subjects having the C/C genotype (“Healthy C/C”); HCV-infected subjects having the C/C genotype (“HCV C/C”); healthy subjects having the C/T genotype (“Healthy C/T”); HCV-infected subjects having the C/T genotype (“HCV C/T”); healthy subjects having the T/T genotype (“Healthy T/T”); and HCV-infected subjects having the T/T genotype (“HCV T/T”). In FIG. 5C, data are plotted showing percent methylation for PM-02 in healthy subjects (“Healthy”) and HCV-infected subjects (“HCV Infected”).

FIG. 6 is a plot showing percent treatment failure by treatment duration and methylation level for CpG PM-02. HCV-infected subjects were treated with a direct-acting antiviral regiment for 8, 12, or 24 weeks. Data are plotted showing percent treatment failure for subjects having less than or equal to 40% methylation at CpG PM-02 (“Methylation ≦40%) and subjects having greater than 40% methylation at PM-02 (“Methylation >40%”).

### DETAILED DESCRIPTION OF THE INVENTION

The present methods can include administering therapeutic agent 1 to a subject. Therapeutic agent 1 is Compound 1 or a pharmaceutically acceptable salt thereof. Therapeutic agent 1 is a direct acting antiviral agent. A direct acting antiviral regimen can include therapeutic agent 1.

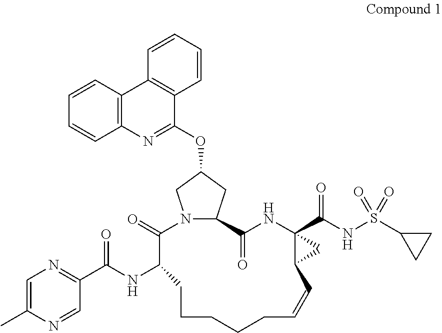 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

Compound 1 is also known as (2R,6S,13aS,14aR,16aS,Z)—N-(cyclopropylsulfonyl)-6-(5-methylpyrazine-2-carboxamido)-5,16-dioxo-2-(phenanthridin-6-yloxy)-1,2,3,5,6,7,8,9,10,11,13a,14,14a,15,16,16a-hexadecahydrocyclopropa[e]pyrrolo[1,2-a][1,4]diazacyclopentadecine-14a-carboxamide. Compound 1 is a potent HCV protease inhibitor. The synthesis and formulation of Compound 1 are described in U.S. Patent Application Publication No. 2010/0144608, U.S. Provisional Application Ser. No. 61/339,964 filed on Mar. 10, 2010, and U.S. Patent Application Publication No. 2011/0312973 filed on Mar. 8, 2011. All of these applications are incorporated herein by reference in their entireties. Therapeutic agent 1 includes various salts of Compound 1. As non-limiting examples, therapeutic agent 1 may be administered in a total daily dosage amount of from 50 mg to 300 mg, preferably from 150 mg to 250 mg, and includes, but is not limited to, for example, 50 mg, 75 mg, 100 mg, 125 mg, 150 mg, 175 mg, 200 mg, 225 mg, 250 mg and suitable amounts there between. In some embodiments, therapeutic agent 1 is administered in a total daily dosage amount of 250 mg. In some embodiments, therapeutic agent 1 is administered in a total daily dosage amount of 150 mg. The total daily dosage amount may be administered in one or more dosage forms and at one or more times daily. The total daily dosage amount may be administered once daily, twice daily, three times daily or at another frequency. For example, the total daily dosage amount may be divided between two dosage forms which are taken at the same time or taken at different times during the day, thereby resulting in twice daily administration.

In some embodiments, ritonavir or another inhibitor of cytochrome P-450 (such as cobicistat) is administered in combination with therapeutic agent 1 to improve the pharmacokinetics of compound 1. In some embodiments, ritonavir is administered at a daily dose of 50 mg to 400 mg, preferably 100 mg. In some embodiments, therapeutic agent 1 is co-administered with the cytochrome 450 inhibitor. For instance, therapeutic agent 1 can be administered, for example and without limitation, concurrently with or sequentially with the cytochrome 450 inhibitor. Therapeutic agent 1 can be administered immediately before or after the administration of the cytochrome 450 inhibitor. A short delay or time gap between the administration of therapeutic agent 1 and the cytochrome 450 inhibitor is also contemplated.

Therapeutic agent 1 can be co-formulated with the cytochrome 450 inhibitor in a single dosage form. Non-limiting examples of suitable dosage forms include liquid or solid dosage forms. The dosage form may be a solid dosage form described in U.S. Publication No. 2011/0312973, entitled “Solid Compositions”, the entire content of which is incorporated herein by reference. For example, the dosage form may be a solid dosage form in which therapeutic agent 1 is molecularly dispersed in a matrix which comprises a pharmaceutically acceptable water-soluble polymer and a pharmaceutically acceptable surfactant. The cytochrome 450 inhibitor can also be molecularly dispersed in the matrix, or formulated in different form(s). As a non-limiting alternative, therapeutic agent 1 and the cytochrome 450 inhibitor can be formulated in two different dosage forms which can be provided as a combination to a subject.

The present methods can include administering therapeutic agent 2 to a subject. Therapeutic agent 2 is Compound 2 or a salt thereof.

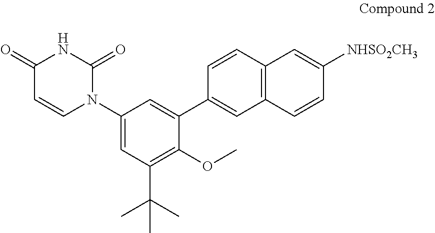

Compound 2 is also known N-(6-(3-tert-butyl-5-(2,4-dioxo-3,4-dihydropyrimidin-1(2H)-yl)-2-methoxyphenyl)naphthalen-2-yl)methanesulfonamide. As described in, for example, International Publication No. WO2009/039127, therapeutic agent 2 includes various salts of Compound 2, such as sodium salts, potassium salts, and choline salts. Therapeutic agent 2 also includes crystalline forms of Compound 2 and its salts such as solvate, hydrate, and solvent-free crystalline forms of Compound 2 and its salts. Compositions comprising therapeutic agent 2 can be prepared as described in, for example, International Publication No. WO2009/039127 which is incorporated by reference herein. Therapeutic agent 2 is a direct acting antiviral agent. A direct acting antiviral regimen can include therapeutic agent 2.

Therapeutic agent 2 may be administered as a free acid, salt or particular crystalline form of Compound 2. In some embodiments, therapeutic agent 2 is administered as a sodium salt. Therapeutic agent 2 may be administered in any suitable amount such as, for example, in doses of from 5 mg/kg to 30 mg/kg. As non-limiting examples, therapeutic agent 2 may be administered in a total daily dosage amount of from 300 mg to 1800 mg, or from 400 mg to 1600 mg, or from 600 mg to 1800 mg, or from 800 mg to 1600 mg or any amounts there between. In some embodiments, the total daily dosage amount for therapeutic agent 2 is 500 mg. In some embodiments, the total daily dosage amount for therapeutic agent 2 is 600 mg. In some embodiments, the total daily dosage amount for therapeutic agent 2 is 800 mg. In some embodiments, the total daily dosage amount for therapeutic agent 2 is 1200 mg. In some embodiments, the total daily dosage amount for therapeutic agent 2 is 1600 mg. The total daily dosage amount may be administered in one or more dosage forms and at one or more times daily. The total daily dosage amount may be administered once daily, twice daily, three times daily or at another frequency. For example, the total daily dosage amount may be divided between two dosage forms which are taken at the same time or taken at different times during the day, thereby resulting in twice daily administration.

The present methods can include administering therapeutic agent 3 or a salt thereof to a subject. Therapeutic agent 3 is compound 3 or a salt thereof.

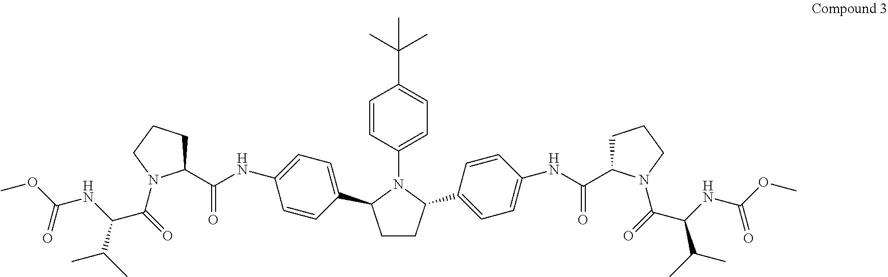

Compound 3 is also known as dimethyl (2S,2′S)-1,1′-((2S,2′S)-2,2′-(4,4′-((2S,5S)-1-(4-tert-butylphenyl)pyrrolidine-2,5,diyl)bis(4,1-phenylene))bis(azanediyl)bis(oxomethylene)bis(pyrrolidine-2,1-diyl)bis(3-methyl-1-oxobutane-2,1-diyl)dicarbamate. Compound 3 can be prepared as described in, for example, U.S. Publication No. 2010/0317568, which is incorporated herein by reference. Therapeutic agent 3 is a direct acting antiviral agent. A direct acting antiviral regimen can include therapeutic agent 3.

Therapeutic agent 3 may be administered as a free acid, or a salt form. Therapeutic agent 3 may be administered in any suitable amount such as, for example, in doses of from 0.1 mg/kg to 200 mg/kg body weight, or from 0.25 mg/kg to 100 mg/kg, or from 0.3 mg/kg to 30 mg/kg. As non-limiting examples, therapeutic agent 3 may be administered in a total daily dose amount of from 5 mg to 300 mg, or from 25 mg to 200 mg, or from 25 mg to 50 mg or any amounts there between. In some embodiments, the total daily dosage amount for therapeutic agent 3 is 25 mg.

Therapeutic agent 3 can be co-formulated with therapeutic agent 1 and/or a cytochrome 450 inhibitor in a single dosage form. Non-limiting examples of suitable dosage forms include liquid or solid dosage forms.

The direct-acting antiviral regimen also can be co-administered with ribavirin, or a pro-drug thereof, in the same or separate pharmaceutical compositions. Ribavirin may include any suitable form or formulation of ribavirin. Exemplary formulations of ribavirin include COPEGUS®, REBETOL® and RIBASPHERE®. An exemplary pro-drug of ribavirin is taribavirin having the chemical name of 1-β-D-ribofuranosyl-1,2,4-triazole-3-carboxamidine. Ribavirin and taribavirin may be administered in accordance with ribavirin and taribavirin administration well known in the art. In some embodiments, COPEGUS® or REBETOL® is administered in a daily dosage amount of from 500 mg to 1500 mg in one dose or in divided doses. In some embodiments, COPEGUS® or REBETOL® is administered in a daily dosage amount of 800 mg. In some embodiments, REBETOL® is administered in a daily dosage amount of 1000 mg. In some embodiments, COPEGUS® or REBETOL® is administered in a daily dosage amount of 1200 mg. In some embodiments, REBETOL® is administered in a daily dosage amount of 1400 mg. Suitable dosages of ribavirin are dependent on the weight of the subject, for example 1000-1200 mg. Suitable total daily dosages of ribavirin include, but are not limited to 400 mg to 1400 mg a day, alternatively 800 mg to 1400 mg per day, alternatively 400 mg to 1200 mg, alternatively 800 mg to 1200 mg.

Current treatment of HCV includes a course of treatment of interferon, e.g. pegylated interferon (e.g., pegylated interferon-alpha-2a or pegylated interferon-alpha-2b, such as PEGASYS by Roche, or PEG-INTRON by Schering-Plough) and the antiviral drug ribavirin (e.g., COPEGUS by Roche, REBETOL by Schering-Plough, or RIBASPHERE by Three Rivers Pharmaceuticals). The treatment often lasts for 24-48 weeks, depending on hepatitis C virus genotype. Other interferons include, but are not limited to, interferon-alpha-2a (e.g., Roferon-A by Roche), interferon-alpha-2b (e.g., Intron-A by Schering-Plough), and interferon alfacon-1 (consensus interferon) (e.g., Infergen by Valeant). Less than 50% of patients with chronic HCV infection with genotype 1 virus respond to this therapy. Further, interferon therapy has many side effects that hinder patient compliance and results in premature discontinuation of the treatment.

The interferon/ribavirin-based treatment may be physically demanding, and can lead to temporary disability in some cases. A substantial proportion of patients will experience a panoply of side effects ranging from a “flu-like” syndrome (the most common, experienced for a few days after the weekly injection of interferon) to severe adverse events including anemia, cardiovascular events and psychiatric problems such as suicide or suicidal ideation. The latter are exacerbated by the general physiological stress experienced by the patients. Ribavirin also has a number of side effects, including, anemia, high pill burden (e.g. 5-6 pills a day split BID) and teratogenicity restricting use in women of childbearing age.

Various measures may be used to express the effectiveness of the present methods of HCV treatment. One such measure is rapid virological response (RVR), meaning that HCV is undetectable in the subject after 4 weeks of treatment, for example, after 4 weeks of administration of two or more of DAAs and ribavirin. Another measure is early virological response (EVR), meaning that the subject has >2 log₁₀ reduction in viral load after 12 weeks of treatment. Another measure is complete EVR (cEVR), meaning the HCV is undetectable in the serum of the subject after 12 weeks of treatment. Another measure is extended RVR (eRVR), meaning achievement of RVR and cEVR, that is, HCV is undetectable at week 4 and 12. Another measure is the presence or absence of detectable virus at the end of therapy (EOT). Another measure is (SVR), which, as used herein, means that the virus is undetectable at the end of therapy and for at least 8 weeks after the end of therapy (SVR8); preferably, the virus is undetectable at the end of therapy and for at least 12 weeks after the end of therapy (SVR12); more preferably, the virus is undetectable at the end of therapy and for at least 16 weeks after the end of therapy (SVR16); and highly preferably, the virus is undetectable at the end of therapy and for at least 24 weeks after the end of therapy (SVR24). SVR24 is often considered as a functional definition of cure; and a high rate of SVR at less than 24 week post-treatment (e.g., SVR8 or SVR12) can be predictive of a high rate of SVR24. Likewise, a high rate of SVR at less than 12 week post-treatment (e.g., SVR4 or SVR8) can be predictive of a high rate of SVR12. A high rate of undetectable virus at EOT (e.g., at week 8 or week 12) can also be indicative of a significant rate of SVR12 or SVR24.

In at least one aspect of the present invention, the effectiveness of the present methods of HCV treatment can be predicted using methylation status of a CpG island within a promoter region of the IL28B gene.

Thus, in certain aspects, the present invention provides methods of predicting an adequate clinical outcome of a patient infected with HCV, comprising detecting the methylation status of a CpG island within a promoter region of the IL28B gene in a sample obtained from the patient. In some embodiments, a methylation level of a CpG island within a promoter region of the IL28B gene in the sample that is greater than a pre-determined control level predicts a failure to achieve sustained response to treatment with an interferon-containing regimen. The pre-determined control level can be a range or a specific value. In some embodiments, the pre-determined control level is a percentage of methylation. In some embodiments, the pre-determined control level is 40%. In some embodiments, the pre-determined control level is 45%. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor, an HCV polymerase inhibitor, and/or an HCV NS5A inhibitor. The direct-acting antiviral regimen can comprise therapeutic agent 1; therapeutic agent 2; and/or therapeutic agent 3.

In other aspects, an HCV-infected patient can be treated with a direct-acting antiviral regimen. Prior to the initiation of treatment with the direct-acting antiviral regimen, a sample is obtained from the patient to establish a methylation level of a CpG island within a promoter region of the IL28B gene. In certain embodiments, the patient is treated with the direct-acting antiviral regimen when the methylation level is greater than a pre-determined control level. The pre-determined control level can be a range or a specific value. In some embodiments, the pre-determined control level is a percentage of methylation. In some embodiments, the pre-determined control level is 40%. In some embodiments, the pre-determined control level is 45%. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor, an HCV polymerase inhibitor, and/or an HCV NS5A inhibitor. The direct-acting antiviral regimen can comprise therapeutic agent 1; therapeutic agent 2; and/or therapeutic agent 3.

In certain other aspects, the present invention provides methods of predicting an inadequate clinical outcome of a patient infected with HCV, comprising detecting the methylation status of a CpG island within a promoter region of the IL28B gene in a sample obtained from the patient. In some embodiments, a methylation level of a CpG island within a promoter region of the IL28B gene in the sample that is less than a pre-determined control level predicts a inadequate sustained response to treatment with an interferon-containing regimen. The pre-determined control level can be a range or a specific value. In some embodiments, the pre-determined control level is a percentage of methylation. In some embodiments, the pre-determined control level is 40%. In some embodiments, the pre-determined control level is 45%. The pre-determined control level can be determined empirically, such as, by obtaining a mean expression level from a population of subjects. The population of subjects can be a population of healthy subjects or a population of HCV-infected subjects. The direct-acting antiviral regimen can comprise an HCV protease inhibitor, an HCV polymerase inhibitor, and/or an HCV NS5A inhibitor. The direct-acting antiviral regimen can comprise therapeutic agent 1; therapeutic agent 2; and/or therapeutic agent 3.

In certain aspects, the present invention provides methods of diagnosing a patient infected with HCV, comprising detecting the methylation status of a CpG island within a promoter region of the IL28B gene in a sample obtained from the patient. In some embodiments, a methylation level of a CpG island within a promoter region of the IL28B gene in the sample that is greater than a pre-determined control level. The pre-determined control level can be a range or a specific value. In some embodiments, the pre-determined control level is a percentage of methylation. The pre-determined control level can be determined empirically, such as, by obtaining a mean expression level from a population of subjects. The population of subjects can be a population of healthy subjects or a population of HCV-infected subjects.

It was unexpectedly discovered that patients with an inadequate response following treatment with pegylated interferon plus ribavirin for 48 weeks (SOC) had higher levels of methylation of a CpG island within the IL28B promoter region as compared to patients that showed an adequate response following treatment with SOC. Thus, methylation levels of a CpG island within the IL28B promoter region can be used as a biomarker for individuals that may relapse or otherwise inadequately respond to treatment. Such information can be used to guide therapy.

In some embodiments, an HCV-infected patient presenting with greater than 40% methylation of a CpG island within the IL28B promoter region can be treated with a direct-acting antiviral regimen designed to effectively treat such patients. In some embodiments, the CpG island can be SEQ ID NO:2 or SEQ ID NO:3. In some embodiments, the direct-acting antiviral regimen may comprise at least two DAAs. Each DAA can be selected from, for example, HCV protease inhibitors, HCV polymerase inhibitors, or HCV NS5A inhibitors.

In some embodiments, an HCV-infected patient presenting with greater than 45% methylation of a CpG island within the IL28B promoter region can be treated with a direct-acting antiviral regimen designed to effectively treat such patients. In some embodiments, the CpG island can be SEQ ID NO:2 or SEQ ID NO:3. In some embodiments, the direct-acting antiviral regimen may comprise at least two DAAs. Each DAA can be selected from, for example, HCV protease inhibitors, HCV polymerase inhibitors, or HCV NS5A inhibitors.

It was unexpectedly discovered that HCV-infected patients had higher levels of methylation of at least one CpG island within the IL28B promoter region as compared to healthy subjects. Thus, methylation levels of a CpG island within the IL28B promoter region can be used as a biomarker for individuals that are infected with HCV. Such information can be used to confirm or establish diagnosis.

It was unexpectedly discovered that patients with an inadequate response following treatment with a direct-acting antiviral regimen for 8 weeks had higher levels of methylation of a CpG island within the IL28B promoter region as compared to patients that showed an adequate response following treatment with a direct-acting antiviral regimen for 8 weeks. Thus, methylation levels of a CpG island within the IL28B promoter region can be used as a biomarker for individuals that may relapse or otherwise inadequately respond to treatment with a direct-acting antiviral regimen. Such information can be used to guide therapy. In particular, such information can be used to select a duration of therapy for a direct-acting antiviral regimen.

In some embodiments, an HCV-infected patient presenting with greater than 40% methylation of a CpG island within the IL28B promoter region can be treated with a direct-acting antiviral regimen for more than 8 weeks. In some embodiments, the treatment duration can be 12 weeks. In some embodiments, the CpG island can be SEQ ID NO:2. In some embodiments, the direct-acting antiviral regimen may comprise at least two DAAs. Each DAA can be selected from, for example, HCV protease inhibitors, HCV polymerase inhibitors, or HCV NS5A inhibitors.

In some embodiments, an HCV-infected patient presenting with less than or equal to 40% methylation of a CpG island within the IL28B promoter region can be treated with a direct-acting antiviral regimen for no more than 8 weeks. In some embodiments, the CpG island can be SEQ ID NO:2. In some embodiments, the direct-acting antiviral regimen may comprise at least two DAAs. Each DAA can be selected from, for example, HCV protease inhibitors, HCV polymerase inhibitors, or HCV NS5A inhibitors.

Certain aspects of the present invention comprise treatment with a direct-acting antiviral regimen. The direct-acting antiviral regimen can comprise, for example, one or more DAAs. For instance, the direct-acting antiviral regimen can comprise a combination of two or more DAAs. The combination of two or more DAAs can be a combination of at least one HCV protease inhibitor and at least one HCV polymerase inhibitor (e.g., a combination of at least one HCV protease inhibitor and at least one non-nucleoside polymerase inhibitor, or a combination of at least one HCV protease inhibitor and at least one nucleoside or nucleotide polymerase inhibitor, or a combination of at least one HCV protease inhibitor, at least one nucleoside or nucleotide polymerase inhibitor and at least one non-nucleoside inhibitor). For another instance, the combination of two or more DAAs can be a combination of at least one HCV protease inhibitor and at least one HCV NS5A inhibitor. For still another instance, the combination of two or more DAAs can be a combination of at least one HCV protease inhibitor, at least one HCV polymerase inhibitor, and at least one HCV NS5A inhibitor. For another instance, the combination of two or more DAAs can be a combination of at least two HCV polymerase inhibitors (e.g., a combination of at least two nucleoside polymerase inhibitors, or a combination of at least one nucleoside or nucleotide polymerase inhibitor and at least one non-nucleoside or nucleotide polymerase inhibitor, or a combination of at least two non-nucleoside polymerase inhibitors). For another instance, the combination of two or more DAAs can be a combination of at least two HCV protease inhibitors. For another instance, the combination of two or more DAAs can be a combination of at least two HCV NS5A inhibitors. For another instance, the combination of two or more DAAs can be a combination of at least one HCV polymerase inhibitor and at least one NS5A inhibitor (e.g., a combination of at least one HCV NS5A inhibitor and at least one non-nucleoside or nucleotide polymerase inhibitor, or a combination of at least one HCV NS5A inhibitor and at least one nucleoside or nucleotide polymerase inhibitor, or a combination of at least one HCV NS5A inhibitor, at least one nucleoside or nucleotide polymerase inhibitor and at least one non-nucleoside polymerase inhibitor).

In one particular embodiment, a direct-acting antiviral regimen is administered to an HCV-infected patient who has been tested for methylation of a CpG island within a promoter region of the IL28B gene. The direct-acting antiviral regimen comprises a combination of therapeutic agent 1 (or a salt thereof), therapeutic agent 2 (or a salt thereof), and therapeutic agent 3 (or a salt thereof). Therapeutic agent 1 (or a salt thereof) can be co-administered or co-formulated with ritonavir and therapeutic agent 3 (or a salt thereof). For example, the direct-acting antiviral regimen can comprise therapeutic agent 1 (or a salt thereof) dosed at 150 mg QD, therapeutic agent 2 (or a salt thereof) dosed at 400 mg BID, therapeutic agent 3 (or a salt thereof) dosed at 25 mg QD, ritonavir dosed at 100 mg QD, and, optionally, ribavirin dosed once or twice daily.

Certain aspects of the present invention comprise treatment of an HCV-infected patient with a direct-acting antiviral regimen. The patient being treated can be a treatment naïve patient, a treatment experienced patient, including, but not limited to, a relapser, an interferon partial responder, an interferon non-responder (e.g., a null responder), or a patient unable to take interferon. The patient may be infected with, for example and without limitation, HCV genotype 1, such as HCV genotype 1a or HCV genotype 1b; or HCV genotype 2 or 3. The treatment may also be effective against other HCV genotypes.

In some of the methods described herein, it is desirable to determine the methylation status of DNA present in a sample. In some embodiments, the sample is a body tissue or body fluid obtained from a human subject (e.g., an HCV-infected patient). In some cases, determination of the methylation status of DNA requires isolation of nucleic acid from a biological sample, such as a cell, biological fluid, or tissue sample. Nucleic acids, including DNA, can be isolated using any suitable technique known in the art. For example, phenol-based extraction is a common method for isolation of DNA.

Detection and quantification of methylation can be achieved by any one of a number of methods well known in the art. Such methods may involve enzymatic digestion of DNA sequences, amplification of DNA sequences, and/or detection of methylation-associated proteins. Examples of methods for the determination of methylation status include, but are not limited to, bisulfite conversion followed by methylation sensitive PCR (MSP) and variations thereof; allele-specific bisulfite sequencing; bisulfite pyrosequencing; whole genome bisulfite sequencing; and HpaII tiny fragment Enrichment by Ligation-mediated PCR (“HELP”) assay. Additional details of various techniques for detection and quantification of methylation of CpG islands can be found in U.S. Pat. Nos. 6,605,432; 8,029,996; 8,242,243; and 8,361,724; and US Publication Nos. 20110046009 and 20120149593, all of which are incorporated by reference in their entireties.

For example, the extent of methylation of a CpG island can be quantified using bisulfite conversion and subsequent analysis. Treatment of DNA with bisulfite converts non-methylated cytosine residues to uracil. The bisulfite-treated DNA can then be analyzed by various techniques known to those of skill in the art. Methods of analysis include, but are not limited to, MSP; methylation-sensitive single-nucleotide primer extension (MS-SnuPE); microarray based methods such as those using methylation-specific probes bound to chips or beads; direct sequencing; and pyrosequencing.

In some embodiments, the methods according to the present invention comprise amplification of a promoter region of the IL28B gene. In some embodiments, the methods according to the present invention comprise amplification of SEQ ID NO:1, SEQ ID NO:2, and/or SEQ ID NO:3.

The sequence of CpG islands and methylation sites (methylation sites are underlined) within the promoter region of the IL28B gene include:

CGCAGACTCGAGCCCGG
(SEQ ID NO: 1; PM-01)
CCGCCCCCTCTGGCAGCACGGAAACCTCCACG
(SEQ ID NO: 2; PM-02)
GGCGTCACAAACCCCGGAGAGCGG
(SEQ ID NO: 3; PM-03)

In further aspects, the present invention provides an article of manufacture comprising: packaging material containing a direct-acting antiviral composition effective to treat a Hepatitis C virus infection; and a label indicating that the composition can be used to treat Hepatitis C virus infection in a patient having a methylation level for a CpG island within a promoter region of an IL28B gene that is amenable to treatment with the composition. For example, the label can indicate that the composition can be used to treat Hepatitis C virus infection in a patient having a methylation level for a CpG island within a promoter region of an IL28B gene that is greater than a pre-determined control level. The pre-determined control level can be a range or a specific value. In some embodiments, the pre-determined control level is a percentage of methylation. In some embodiments, the pre-determined control level is 40%. In some embodiments, the pre-determined control level is 45%. The pre-determined control level can be determined empirically, such as, by obtaining a mean expression level from a population of subjects. The population of subjects can be a population of healthy subjects or a population of HCV-infected subjects. In some embodiments, the label can indicate that the composition can be used to treat Hepatitis C virus infection in a patient having greater than 40% methylation of a CpG island within a promoter region of an IL28B gene. In some embodiments, the label can indicate that the composition can be used to treat Hepatitis C virus infection in a patient having greater than 45% methylation of a CpG island within a promoter region of an IL28B gene. Particular direct-acting antiviral compositions effective to treat a Hepatitis C virus infection include, for example, protease inhibitors, nucleoside or nucleotide polymerase inhibitors, non-nucleoside polymerase inhibitors, NS3B inhibitors, NS4A inhibitors, NS5A inhibitors, NS5B inhibitors, cyclophilin inhibitors, and combinations of any of the foregoing. For example, a composition effective to treat a Hepatitis C virus infection can comprise therapeutic agent 1, therapeutic agent 2, therapeutic agent 3, or a combination thereof. The composition can be co-administered or co-formulated with ritonavir. The composition also can be co-administered with ribavirin.

In yet further aspects, the present invention provides systems for effective treatment of Hepatitis C virus infection, comprising: a measurement of methylation of a CpG island within a promoter region of an IL28B gene for a patient; and a direct-acting antiviral regimen. The direct-acting antiviral regimen can comprise an HCV protease inhibitor, an HCV polymerase inhibitor, and/or an HCV NS5A inhibitor. The systems can further comprise an assay for providing the measurement of the methylation level of a CpG island within a promoter region of an IL28B gene. The systems also can comprise directions whether to administer the direct-acting antiviral regimen to a patient based on the measurement.

### Example 1

### Percent Methylation in Three CpG Islands in the IL28B Promoter in Treatment-Naïve and Treatment-Experienced HCV-Infected Patients

Treatment-naïve patients and treatment-experienced patients were included in the study. The treatment-experienced patients previously received peg-interferon and ribavirin (“SOC”), but did not achieve an adequate sustained response. Subjects included 30 treatment-naïve subjects and 14 treatment-experienced subjects between the ages of 18 and 65.

DNA samples were analyzed for methylation in three CpG islands in the IL28B promoter. The three CpG islands correspond to SEQ ID NO:1 (PM-01), SEQ ID NO:2 (PM02), and SEQ ID NO:3 (PM03).

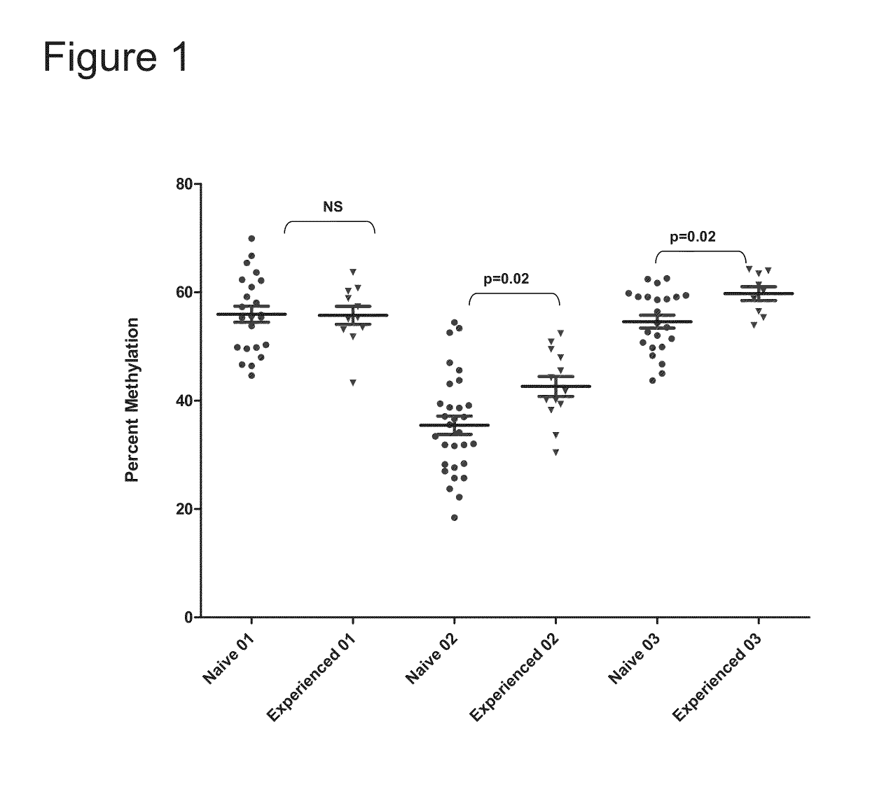

As shown in FIG. 1, analysis of the three CpG islands in the IL28B promoter suggests that subjects who failed SOC treatment as a group may have higher methylation levels in two of three islands. The higher methylation levels may repress expression of the IL28B gene.

### Example 2

### Methylation Status of IL28B PM-02 and Response to SOC after 10 Days of SOC Treatment

Eleven (11) treatment-naïve patients were included in the study. The patients received peg-interferon and ribavirin (“SOC”) for ten (10) days.

DNA samples obtained prior to the initiation of SOC treatment were analyzed for methylation in a CpG island in the IL28B promoter corresponding to SEQ ID NO:2 (PM02). Subjects were grouped based on methylation level of PM02. Six (6) subjects had methylation values below 45% (squares). Five (5) subjects had methylation values above 45% (diamonds).

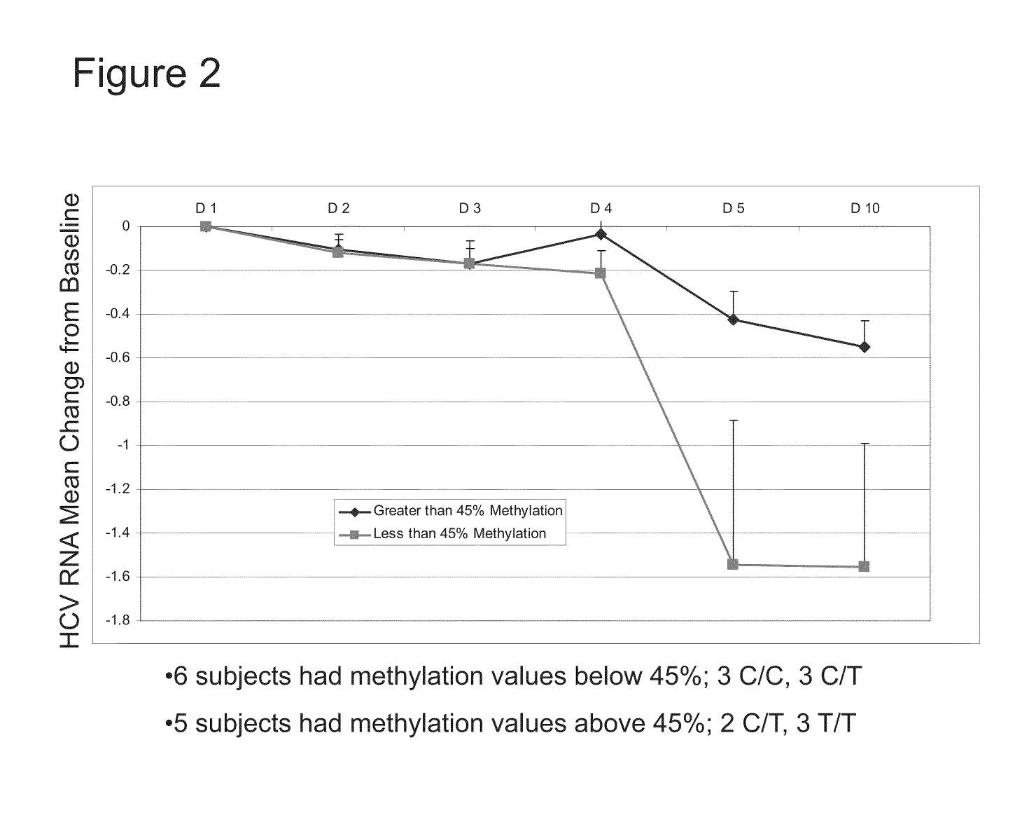

Viral load was assessed on Day 1 (“D1”), Day 2 (“D2”), Day 3 (“D3”), Day 4 (“D4”), Day 5 (“D5”), and Day 10 (“D10”) of treatment. As shown in FIG. 2, the subjects having methylation values less than 45% showed a dramatic reduction in viral load after 10 days of SOC treatment. Conversely, the subjects having methylation values greater than 45% showed minimal reduction in viral load after 10 days of SOC treatment. This analysis suggests that subjects who respond inadequately to SOC treatment as a group may have higher methylation levels in the CpG island of the IL28B promoter corresponding to corresponding to SEQ ID NO:2 (PM02).

### Example 3

### Methylation Status of IL28B PM-02 and Response to Compound 1 after 10 Days of Treatment

Twenty-one (21) treatment-naïve patients were included in the study. The patients received Compound 1 and ritonavir for three days followed by the addition of peginterferon and ribavirin for a total of 12 weeks of combination treatment.

DNA samples obtained prior to the initiation of treatment were analyzed for methylation in a CpG island in the IL28B promoter corresponding to SEQ ID NO:2 (PM02). Subjects were grouped based on methylation level of PM02. Eleven (11) subjects had methylation values below 45% (diamonds). Ten (10) subjects had methylation values above 45% (squares).

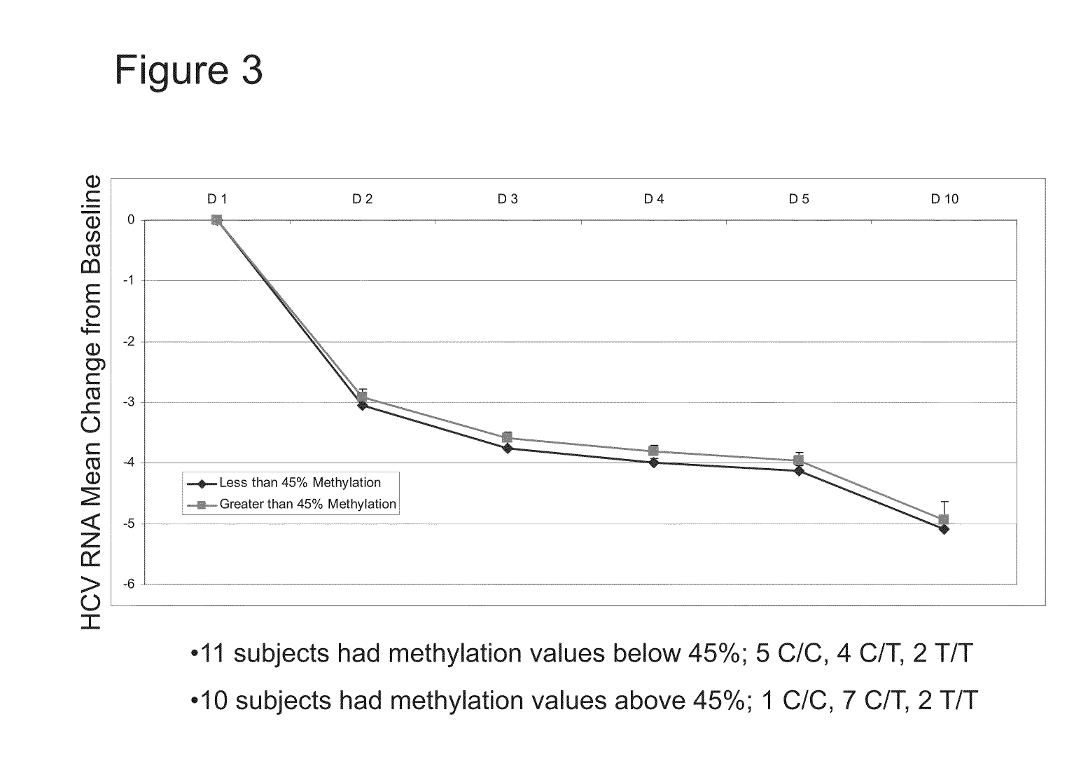

Viral load was assessed on Day 1 (“D1”), Day 2 (“D2”), Day 3 (“D3”), Day 4 (“D4”), Day 5 (“D5”), and Day 10 (“D10”) of treatment. As shown in FIG. 3, both groups showed a dramatic reduction in viral load after 10 days of treatment. This analysis suggests that methylation status of the CpG island of the IL28B promoter corresponding to corresponding to SEQ ID NO:2 (PM02) does not affect response to Compound 1.

### Example 4

### Association with rs12979860 Allele Status

One hundred seventeen (117) subjects infected with GT1 HCV were included in the study: 44 subjects from Example 1, and an additional 73 treatment-naïve subjects from a separate study.

A SNP in the IL-28B gene (rs12979860) was analyzed to determine each patient's genotype. Patients were identified as having the C/C genotype (circles); the C/T genotype (triangles); or the T/T genotype (squares).

DNA samples were analyzed for methylation in three CpG islands in the IL28B promoter. The three CpG islands correspond to SEQ ID NO:1 (PM-01), SEQ ID NO:2 (PM02), and SEQ ID NO:3 (PM03).

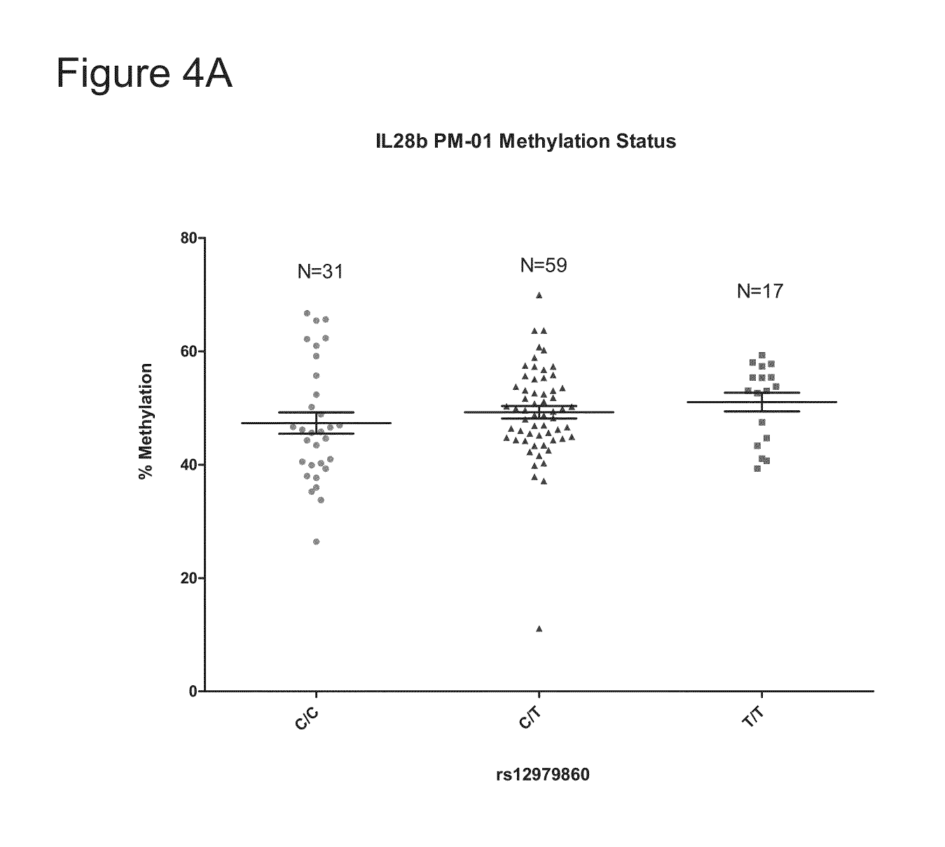

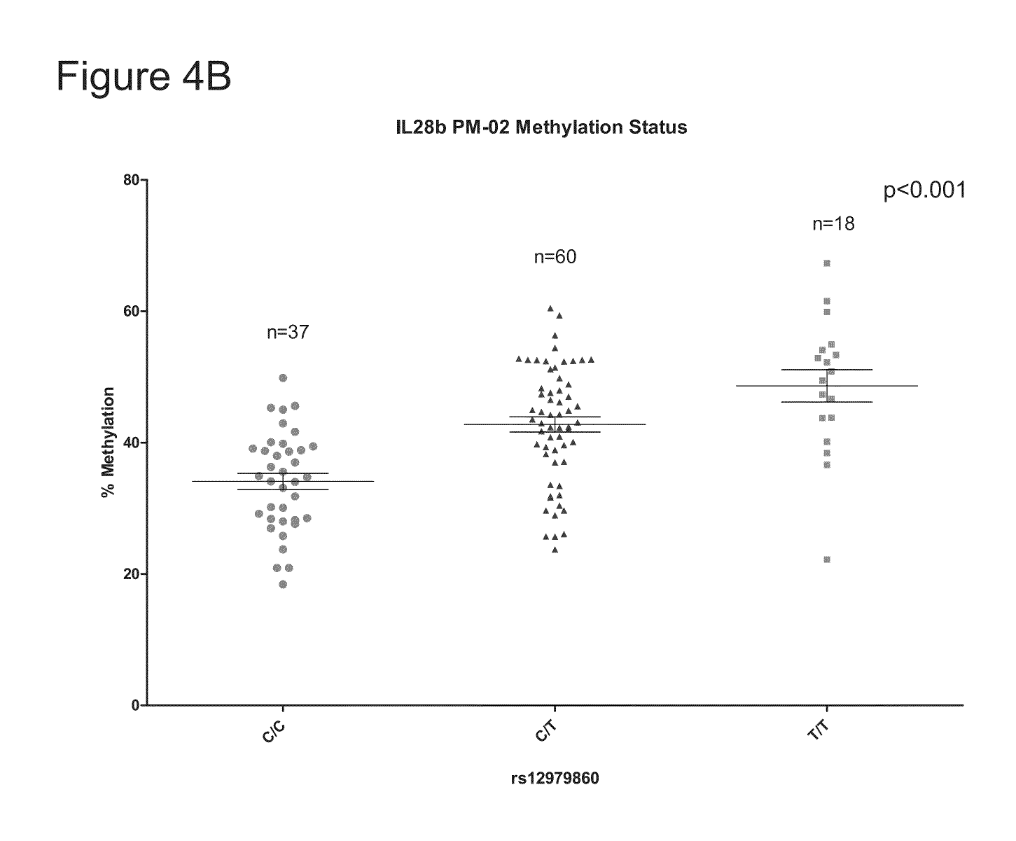

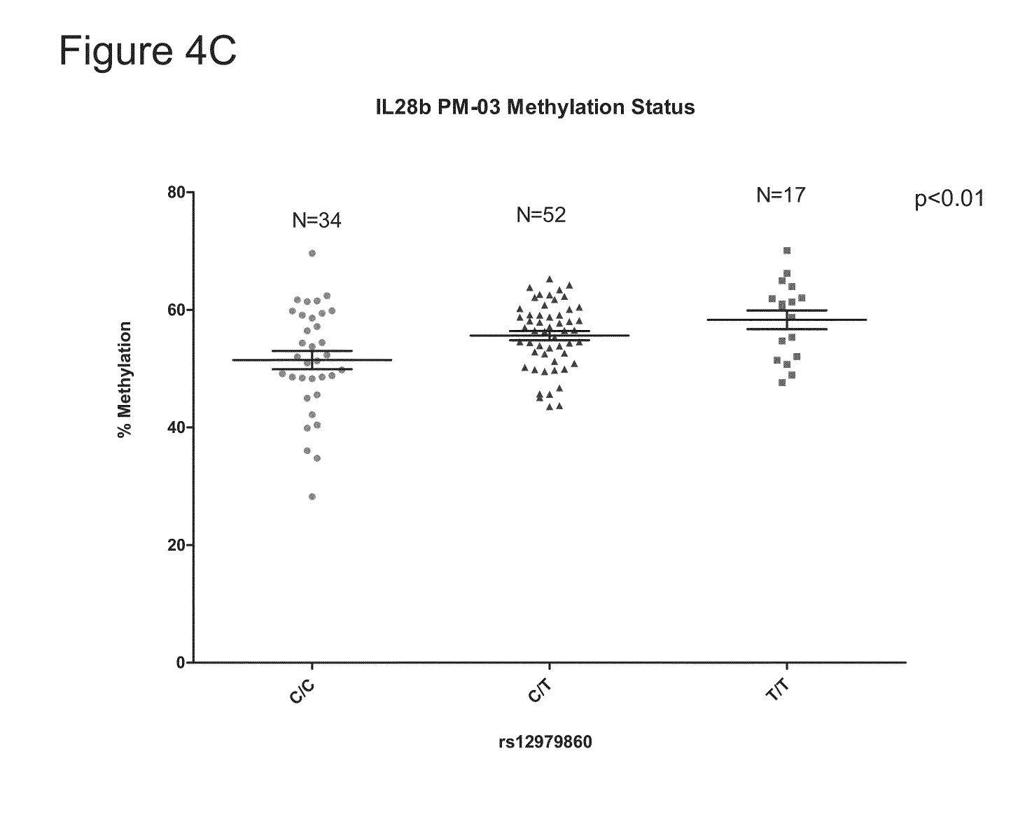

For the analysis of methylation of PM-01, 10 DNA samples failed quality control and were not included. As seen in FIG. 4A, methylation of PM-01 does not correlate with rs12979860 allele status.

For the analysis of methylation of PM-02, 2 DNA samples failed quality control and were not included. As seen in FIG. 4B, methylation of PM-02 correlates with rs12979860 allele status (p<0.001). Patients having the C/C genotype have lower levels of methylation of PM-02 as compared to patients having the C/T or T/T genotype. Patients having the C/T genotype have intermediate levels of methylation of PM-02; between the methylation levels for patients having the C/C or T/T genotype.

For the analysis of methylation of PM-03, 12 DNA samples failed quality control and were not included. As seen in FIG. 4C, methylation of PM-03 correlates with rs12979860 allele status (p<0.01). Patients having the C/C genotype have lower levels of methylation of PM-03 as compared to patients having the C/T or T/T genotype.

These results suggest that methylation status of CpG islands within the promoter region of the IL28B gene can be used as biomarkers for predicting and evaluating treatment response. For example, high levels of methylation of CpG PM-02 or CpG PM-03 could be used to identifying subjects amenable to receiving a direct-acting antiviral regimen.

### Example 5

### Percent Methylation in Three CpG Islands in the IL28B Promoter in Healthy Controls and Treatment-Naïve HCV-Infected Subjects

One hundred fifty three (153) healthy subjects and one hundred twelve (112) treatment-naïve HCV-infected subjects were included in the study.

DNA samples obtained from white blood cells were analyzed for methylation in three CpG islands in the IL28B promoter. The three CpG islands correspond to SEQ ID NO:1 (PM-01), SEQ ID NO:2 (PM02), and SEQ ID NO:3 (PM03).

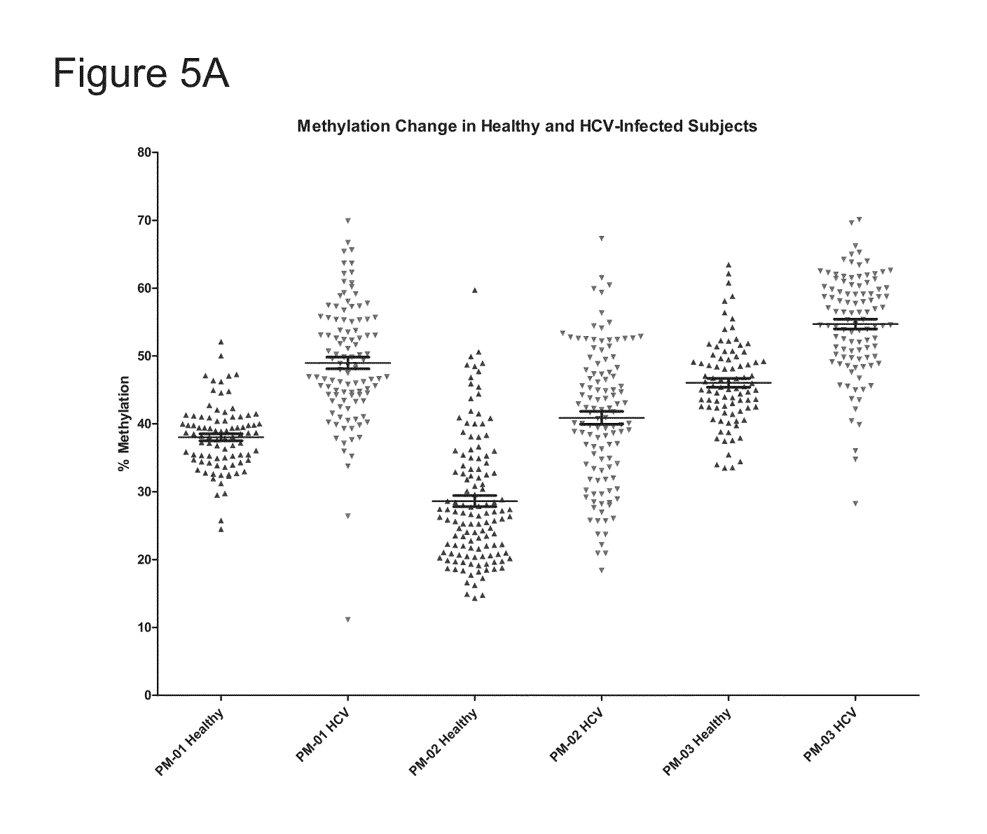

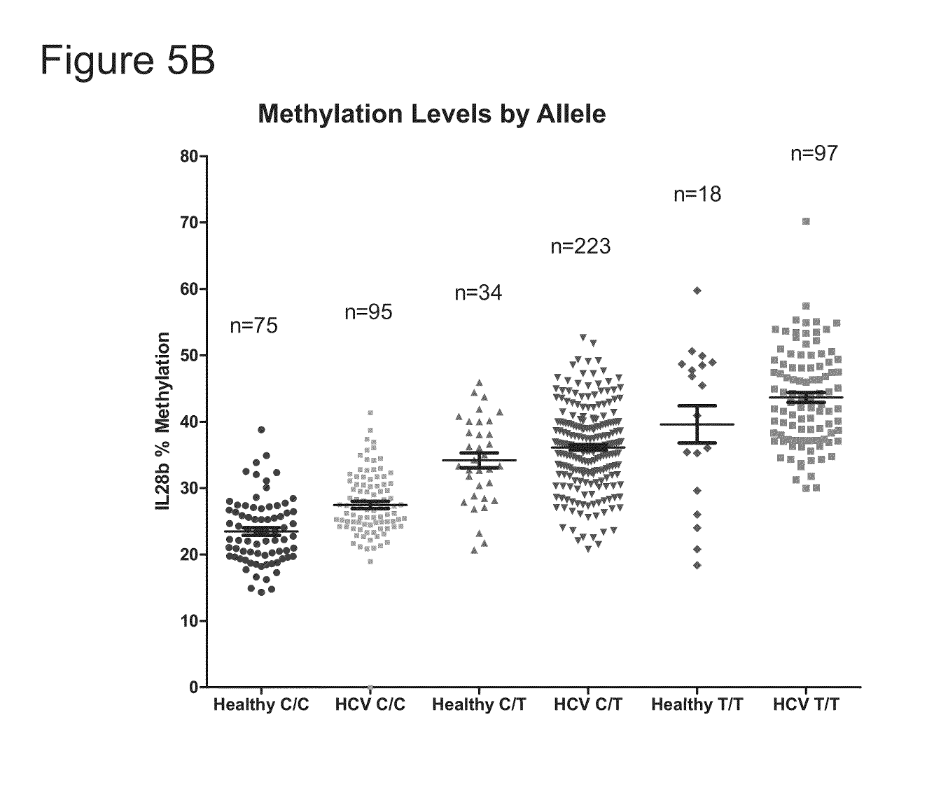

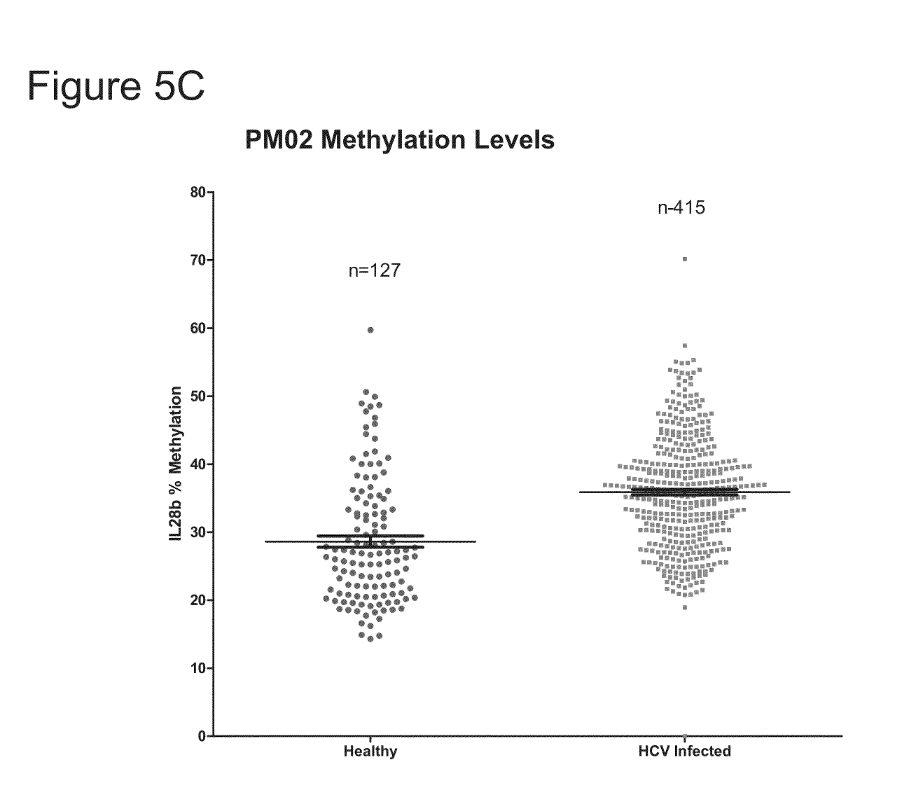

As shown in FIG. 5A, analysis of the three CpG islands in the IL28B promoter reveals that HCV-infected patients have higher methylation levels at each of the three CpG islands as compared to healthy subjects. Thus, IL28B methylation levels are higher in HCV-infected subjects relative to healthy controls.

PM-02 methylation was further analyzed by comparing the methylation level in HCV-infected subjects to healthy subjects as a function of rs12979860 allele status. As shown in FIG. 5B, PM-02 methylation correlates with rs12979860 allele status in both healthy subjects and HCV-infected subjects. These results confirm that CpG island methylation in the IL28B promoter shows a strong association with the IL28B rs12979860 allele status. Healthy patients having the C/C genotype have lower levels of methylation of PM-02 as compared to healthy patients having the C/T or T/T genotype.

PM-02 methylation was further analyzed by comparing the methylation level in HCV-infected subjects to healthy subjects. As shown in FIG. 5C, analysis of PM02 reveals that HCV-infected patients have higher methylation as compared to healthy subjects. Thus, PM02 methylation levels are higher in HCV-infected subjects relative to healthy controls.

### Example 6

### Treatment Failure by Percent Methylation of IL28B PM-02

Four hundred thirty eight (438) treatment-naïve HCV-infected subjects and one hundred thirty three (133) subjects that had failed previous therapy with pegylated interferon and ribavirin (“P/R treatment failures”) were included in the study.

Subjects were treated with a DAA regimen that included Compound 1/ritonavir (“C1/r”), Compound 2 (“C2”), Compound 3 (“C3”), and/or ribavirin (“RBV”) for 8, 12, or 24 weeks. The treatment groups are summarized in Table 1.

TABLE 1

| Population | Treatment-Naïve | | | | | | P/R Treatment Failure | | |
|---|---|---|---|---|---|---|---|---|---|
| Regimen | C1/r | C1/r | C1/r | C1/r | C1/r | C1/r | C1/r | C1/r | C1/r |
| | C2 | C2 | | C2 | C2 | C2 | | C2 | C2 |
| | C3 | | C3 | C3 | C3 | C3 | C3 | C3 | C3 |
| | RBV | RBV | RBV | | RBV | RBV | RBV | RBV | RBV |
| Duration | 8 wks | 12 wks | 12 wks | 12 wks | 12 wks | 24 wks | 12 wks | 12 wks | 24 wks |
| n | 80 | 41 | 79 | 79 | 79 | 80 | 45 | 45 | 43 |
| DNA Samples | 52 | 31 | 48 | 58 | 63 | 52 | 38 | 35 | 38 |
| Analyzed | | | | | | | | | |
| Breakthrough | 10 | 5 | 9 | 6 | 1 | 2 | 5 | 3 | 1 |
| or Relapse | | | | | | | | | |

DNA samples obtained prior to the initiation of the DAA regimen were analyzed for methylation in a CpG island in the IL28B promoter corresponding to SEQ ID NO:2 (PM02). Subjects were grouped based on methylation level of PM02. Three hundred (300) subjects had methylation values less than or equal to 40%. One hundred thirteen (113) subjects had methylation values greater than 40%. Subject were further grouped by treatment duration: 8 weeks, 12 weeks or 24 weeks.

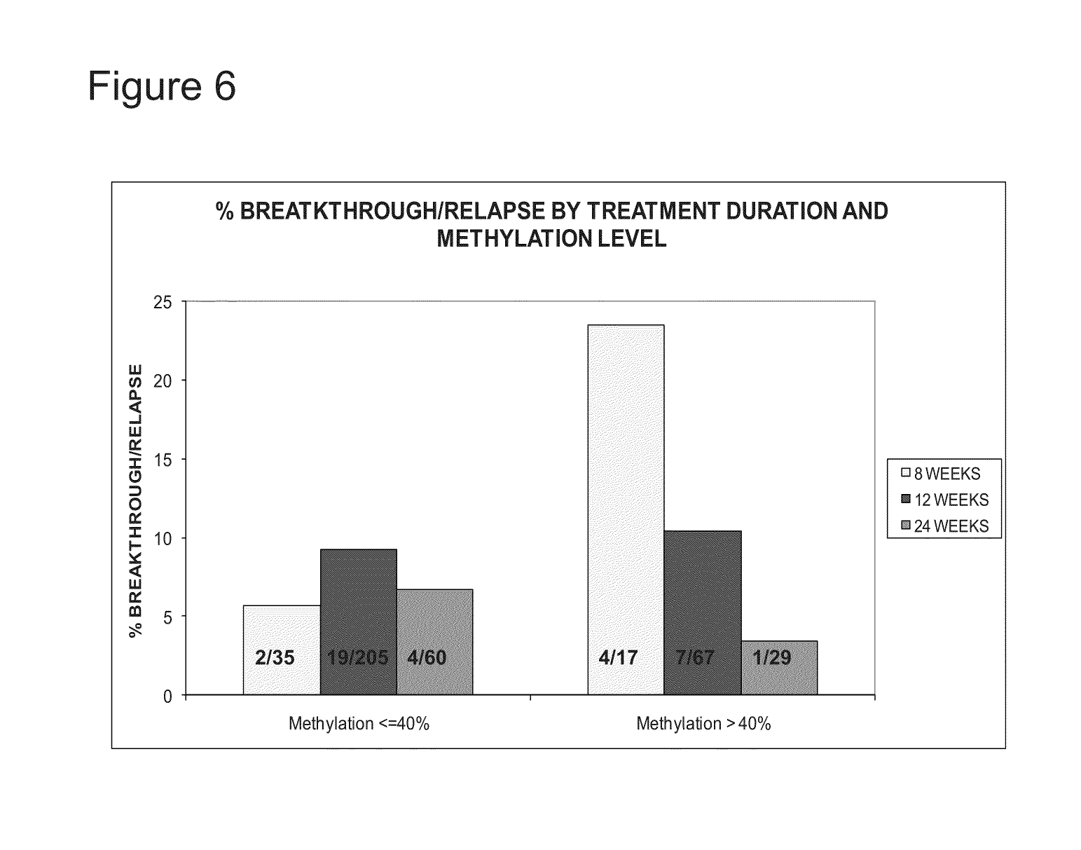

FIG. 6 shows the treatment failure (breakthrough or relapse) rate by treatment duration and methylation level of the CpG island in the IL28B promoter corresponding to SEQ ID NO:2 (PM02).

Higher methylation levels are associated with higher rate of treatment failure in subjects receiving 8 weeks of the DAA regimen. Methylation levels are not associated with treatment failure in subjects receiving 12 or 24 weeks of the DAA regimen. Thus, methylation levels of a CpG island in the IL28B promoter region may be used to select an adequate treatment duration for an HCV-infected patient.

The treatment failure rate for patients that received an 8 week course of a direct-acting antiviral regimen was higher in a group having higher levels of methylation (e.g., greater than 40% methylation) of at least one CpG island within an IL28B promoter region as compared to the group having lower levels of methylation (e.g., less than or equal to 40% methylation). In particular, a patient having greater than 40% methylation of a CpG island in the IL28B promoter region may be selected as a candidate to receive a DAA regimen for more than 8 weeks. For example, a patient having greater than 40% methylation of the CpG island corresponding to SEQ ID NO:2 (PM02) may be selected as a candidate to receive a DAA regimen for 12 or 24 weeks. Conversely, a patient having less than or equal to 40% methylation of a CpG island in the IL28B promoter region may be selected as a candidate to receive a DAA regimen for no more than 8 weeks. For example, a patient having less than or equal to 40% methylation of the CpG island corresponding to SEQ ID NO:2 (PM02) may be selected as a candidate to receive a DAA regimen for 8 weeks.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method for treating a subject infected with Hepatitis C virus with a direct-acting antiviral regimen, the method comprising:
administering the direct-acting antiviral regimen to the subject;
wherein prior to the administration of the direct-acting antiviral regimen a DNA sample from the patient has been tested to determine methylation status of at least one CpG island in an IL28B promoter.

2. The method of claim 1, wherein the CpG island comprises SEQ ID NO:1, SEQ ID NO:2, or SEQ ID NO:3.

3. The method of claim 1, wherein the CpG island comprises SEQ ID NO:2 or SEQ ID NO:3 and the methylation status is greater than 45% methylation.

4. The method of claim 1, wherein the direct-acting antiviral regimen comprises an HCV protease inhibitor, an HCV polymerase inhibitor, and an HCV NS5A inhibitor.

5. The method of claim 1, wherein the direct-acting antiviral regimen comprises therapeutic agent 1, therapeutic agent 2, and therapeutic agent 3.

6. A kit for predicting responsiveness of a subject to treatment of hepatitis C infection, the kit comprising:
an assay for detecting methylation of at least one CpG island in a promoter region of an IL28B gene; and
a written instruction that states a level of hypermethylation of the CpG island that predicts an inadequate response to treatment.

7. The kit of claim 6, wherein the CpG island comprises SEQ ID NO:1, SEQ ID NO:2, or SEQ ID NO:3.

8. The kit of claim 6, wherein the CpG island comprises SEQ ID NO:2 or SEQ ID NO:3 and the level of hypermethylation is greater than 45% methylation.

9. The kit of claim 6, wherein the treatment comprises peg-interferon and ribavirin.

10. A method for detecting a risk of treatment failure in a Hepatitis C infected subject, the method comprising:
(a) providing a sample comprising a promoter region of an IL28B gene from the Hepatitis C infected subject;
(b) detecting methylation status of at least one CpG island in the promoter region of the IL28B gene from the Hepatitis C infected subject, wherein hypermethylation of the CpG island in the promoter region is indicative of a risk for treatment failure.

11. The method of claim 10, wherein the CpG island comprises SEQ ID NO:1, SEQ ID NO:2, or SEQ ID NO:3.

12. The method of claim 10, further comprising administering a direct-acting antiviral regimen to the subject

13. The method of claim 12, wherein the direct-acting antiviral regimen comprises an HCV protease inhibitor, an HCV polymerase inhibitor, and an HCV NS5A inhibitor.

14. The method of claim 12, wherein the direct-acting antiviral regimen comprises therapeutic agent 1, therapeutic agent 2, and therapeutic agent 3.

15. The method of claim 10, wherein the sample is a DNA sample.

16. A method for determining a treatment regimen for a subject infected with hepatitis C as a candidate to receive direct-acting antiviral therapy, comprising:
(a) providing a biological sample from the subject, the sample comprising a promoter region of an IL28B gene;
(b) detecting methylation status of at least one CpG island in the promoter region of the IL28B gene; and
(c) classifying the patient as eligible to receive direct-acting antiviral therapy based on the methylation status of the CpG island.

17. The method of claim 16, wherein the CpG island comprises SEQ ID NO:1, SEQ ID NO:2, or SEQ ID NO:3.

18. The method of claim 16, further comprising administering a direct-acting antiviral regimen to the subject.

19. The method of claim 18, wherein the direct-acting antiviral regimen comprises an HCV protease inhibitor, an HCV polymerase inhibitor, and an HCV NS5A inhibitor.

20. The method of claim 18, wherein the direct-acting antiviral regimen comprises therapeutic agent 1, therapeutic agent 2, and therapeutic agent 3.

21. A method for treating a subject with Hepatitis C infection, the method comprising:
(a) providing a DNA sample from the subject, the sample comprising a non-coding region of an IL28B gene;
(b) determining methylation status of at least one CpG island in the non-coding region of the IL28B gene, wherein hypermethylation of the CpG island in the non-coding region is indicative of the subject being at least partially insensitive to treatment with an interferon-containing regimen; and
(c) administering a direct-acting antiviral regimen to the subject.

22. The method of claim 21, wherein the CpG island comprises SEQ ID NO:1, SEQ ID NO:2, or SEQ ID NO:3.

23. The method of claim 21 wherein the direct-acting antiviral regimen comprises an HCV protease inhibitor, an HCV polymerase inhibitor, and an HCV NS5A inhibitor.

24. The method of claim 21, wherein the direct-acting antiviral regimen comprises therapeutic agent 1, therapeutic agent 2, and therapeutic agent 3.
